(19) United States
(12) Patent Application Publication
(10) Pub. No.: US 2021/0299373 A1
(43) Pub. Date: Sep. 30, 2021

(54) FILTERED RESPIRATION

(71) Applicant: Pankaj Merchia, Boca Raton, FL (US)

(72) Inventor: Pankaj Merchia, Boca Raton, FL (US)

(21) Appl. No.: 17/216,667

(22) Filed: Mar. 29, 2021

Related U.S. Application Data
(60) Provisional application No. 63/001,250, filed on Mar. 28, 2020.
(60) Provisional application No. 63/003,884, filed on Apr. 1, 2020.
(60) Provisional application No. 63/005,196, filed on Apr. 3, 2020.
(60) Provisional application No. 63/015,707, filed on Apr. 27, 2020.
(60) Provisional application No. 62/704,392, filed on May 8, 2020.
(60) Provisional application No. 62/704,689, filed on May 22, 2020.

(51) Int. Cl. A61M 16/00; A61M 16/10; A61M 16/20; A61M 16/08

(52) U.S. Cl. CPC A61M 16/0093; A61M 16/0057; A61M 2205/75; A61M 16/205; A61M 16/0808; A61M 16/1065

(57) ABSTRACT

The present invention is directed to filtered respiration that can occur according to multiple facets. The present invention includes a Continuous Positive Airway Pressure (“CPAP”) system, an adapter, and a CPAP interface. Other inventions disclosed herein relate to the modification of CPAP and BILEVEL-PAP systems into ventilators.

### RELATED APPLICATIONS

The present application claims priority pursuant to 35 U.S.C. § 119 from U.S. Provisional Patent Application No. 63/001,250 filed Mar. 28, 2020, as well as priority from U.S. Provisional Patent Application No. 63/003,884 filed Apr. 1, 2020, as well as priority from U.S. Provisional Patent Application No. 63/005,196 filed Apr. 3, 2020, as well as U.S. Provisional Patent Application No. 63/015,707 filed Apr. 27, 2020, as well as U.S. Provisional Patent Application No. 62/704,392 filed May 8, 2020, as well as U.S. Provisional Patent Application No. 62/704,689 filed May 22, 2020.

### FIELD OF THE INVENTION

The present invention relates to the field of artificial respiration and more specifically to the field of mechanical ventilation.

### BACKGROUND

Obstructive Sleep Apnea (“OSA”) is a common condition where the airway collapses when an individual is sleeping; partial collapse causes snoring. OSA affects about 22% of Men and 17% of women or about 1 in 5 persons (J Thorac Dis. 2015 August; 7(8): 1311-1322). Thus OSA affects about 60 million people in the US. CPAP machines are used by millions of people to treat their obstructive sleep apnea. CPAP machines are currently used in the following system configuration as shown in FIG. 1. A CPAP machine motivator 120a blows room air into tubing 130 pressurizing the air. Tubing carries pressurized air from the CPAP motivator 120a to a patient mask, or other interface 140. A Patient mask covers patient nose and sometimes mouth as well. An exhaust vent on the mask allows exhaled air to be released into room. When a patient exhales, most of its exhaled air goes into the tubing and mask. This exhaled air is then exhausted into the room through the vent on the mask. Limitations to CPAP systems make their use by infected patients potentially unsafe because CPAP machines disperse exhaled air with pressure resulting in a patient's exhaled air being spread to others in their vicinity (e.g. healthcare workers, family members) and onto surfaces in their vicinity more than it would be spread with normal exhalation. Thus, use of CPAP by patients with an infection (e.g. COVID-19) presents an increased risk of spreading infection. Spread of infection is particularly dangerous in hospital settings.

The COVID-19 pandemic has resulted in a shortage of ventilators. Ventilators are essential to provide respiratory assistance to patients with severe COVID-19 disease who are unable to breath by themselves or need positive pressure in their lungs to overcome inflammation and fluid from infection. BilevelPAP machines are similar to ventilators in they provide a higher pressure for inspiration and a lower pressure for expiration with the difference in pressures driving breaths. There are many millions of BiLevPAP machines currently in homes around the US and the world. There are over 50,000 BilevelPAP machines in US hospital (this is similar to the about 70,000 ventilators in US hospitals at the drafting of this document.). Hundreds of thousands of BilevelPAP machines are made and sold each year. Thus, large numbers of BilevelPAP machines are readily available in homes, hospitals, and for purchase.

BilevelPAP machines are currently used in the following system configuration as shown in FIG. 3. A BilevelPAP motivator 120b blows ambient air 180 into tubing 130 pressurizing the air 184. Tubing carries pressurized air from the CPAP motivator 120b to a patient mask or other interface 140. A patient mask covers the patient nose and sometimes the mouth as well. An exhaust vent on mask allows exhaled air to be released into room. When the patient exhales, most of his exhaled air goes into the tubing and mask. This exhaled air is then exhausted into the room through the vent on the mask.

Limitations of BilevelPAP systems make their use as ventilators inadequate because that exhaled air from the patient is dispersed into the room, typically through exhaust vents on the mask, and could infect others in their vicinity (e.g. healthcare workers, family members, caregivers, other patients) and contaminate surfaces around the patient. Also, some patients require additional oxygen supplementation. Furthermore, some patients are unable to protect their airway from oral secretions and other liquids. Some patients are unable to initiate breaths on a regular basis and thus need an external trigger to initiate their breaths (i.e. if the patient does not try to breath, the machine will not automatically increase the pressure to help them breath, say 15 or some other number of times per minute).

### SUMMARY

The present invention is directed to filtered respiration that can occur according to multiple facets. The present invention includes a Continuous Positive Airway Pressure (“CPAP”) system, an adapter, and a CPAP interface. Other inventions disclosed herein relate to the modification of CPAP and BILEVEL-PAP systems into ventilators.

The CPAP system includes a gas motivator, gas channel, respiration interface, and an adapter. The gas motivator is a device that urges gas along a particular vector, and may include any form of air mover or the like. Conventional CPAP blowers are ideal in connection with the present invention. A gas channel connects to the gas motivator and leads pressurized gas to a respiration interface. The gas channel can include, conduit, piping, or any other means of conduction of a gas from one point to another. The typical gas channel includes the piping associated with conventional CPAP equipment; however, the significance of the gas channel is merely that it transports gas in the direction of a user/patient.

A respiration interface includes the delivery means of the gas from the gas motivator. Conventional forms of masks associated with CPAP systems may be utilized with the present invention, so long as they originally or as-modified meet the objectives of the present invention. The point of delivery for CPAP, and related systems, often includes a mask that covers the nose and/or mouth of a user/patient, but other forms of interface may be utilized such as internal tubing, e.g. endotracheal tube, or a laryngeal mask. The respiration interface is the point of delivery of gas to the airway of a user. The airway as discussed herein includes any respiratory channel of a user's body, including nasal, mouth, and tracheal passages. The respiration interface sealingly connects to the airway of the user, meaning that to the best of equipment's (i) intended purpose or (ii) manufacturing tolerances (an exclusive of human cooperation), there is a barrier between the airway of the user and the ambient environment in which the user is located.

The present invention includes a dedicated, filtered passage in an adapter that permits gas exhaled by a user to be cleansed of malicious agents with particular characteristics such as particulate size or chemical composition. The malicious agents of the present invention can include chemicals, viruses, bacteria, mold, or any other agent that can be harmful to those surrounding the user/patient and desirous of blocking. The adapter can be positioned on the interface, the channel, lie therebetween, or occupy any other position that it is in the path of exhaled gas. The adapter includes a respiratory outlet having a filter that obstructs an exclusive passage of exhaled gas from the user to the ambient environment. In certain versions of the present invention, the exclusive passage can be purpose-built and available originally; or in other versions of the present invention, the exclusive passage can exist by virtue of a dedicated effort to seal other passages in any component of the system that might result in exhaled air encountering the ambient environment. By “exclusive passage” it is not meant to be limiting in quantity or construction, but rather it is meant that to the extent that there is a passage from the airway to the ambient environment, it is through the one or more filters of the present invention. The “exclusive passage” can be bifurcated, or take the form of multiple branches, etc. However, the present invention results in a significant reduction in the danger posed by unfiltered exhalant spewed or aerosolized into the ambient environment by a user.

The functionality of the adapter can be integrated as a component, or a stand-alone component. For example, the adapter may be integrated, affixed, included with, or otherwise associated with an interface. The adapter may be integrated, affixed, included with, or otherwise associated with a gas channel. The adapter may be a discrete item added to the gas channel, interface, or therebetween.

These aspects of the invention are not meant to be exclusive. Furthermore, some features may apply to certain versions of the invention, but not others. Other features, aspects, and advantages of the present invention will be readily apparent to those of ordinary skill in the art when read in conjunction with the following description, and accompanying drawings.

### BRIEF DESCRIPTION OF THE DRAWINGS

FIG. 1 is a schematic view of a conventional CPAP system arrangement.

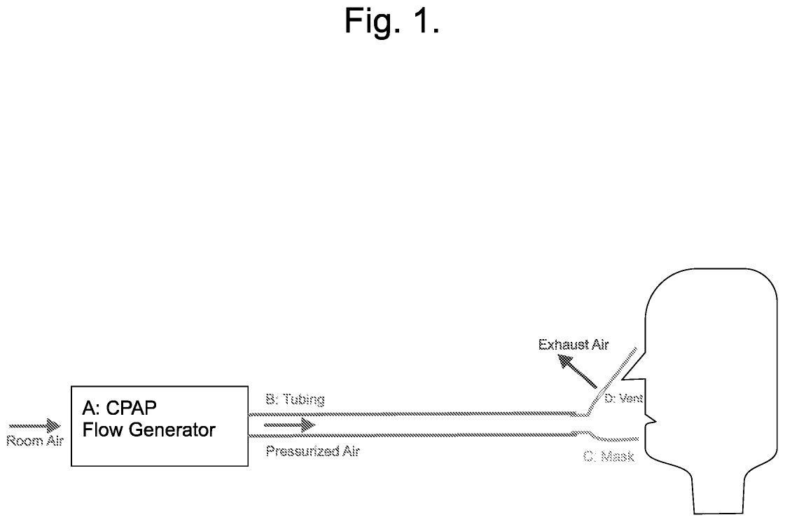

FIG. 2 is a schematic view of the system and process of the present invention.

FIG. 3 is a schematic view of a conventional BILEVEL-PAP system arrangement.

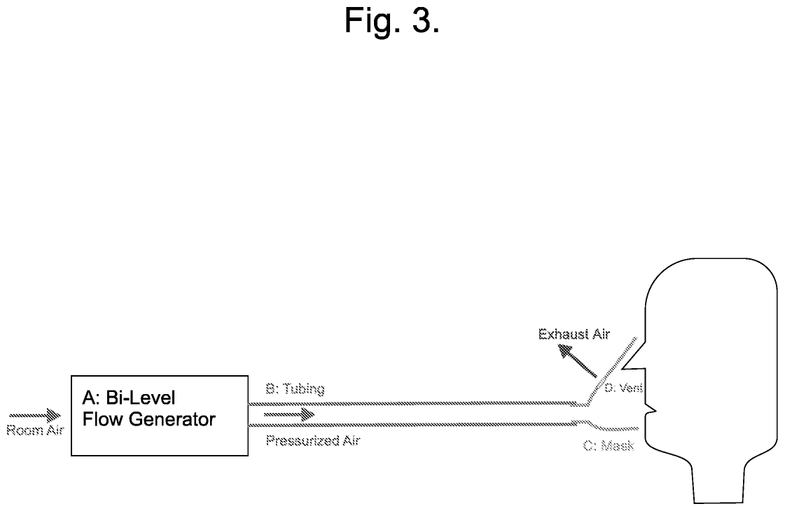

FIG. 4 is a schematic view of the system and process of the present invention.

FIG. 5 is a schematic view of the system and process of the present invention.

FIG. 6 is a schematic view of the system and process of the present invention.

FIG. 7 is a schematic view of the system and process of the present invention.

### DETAILED DESCRIPTION

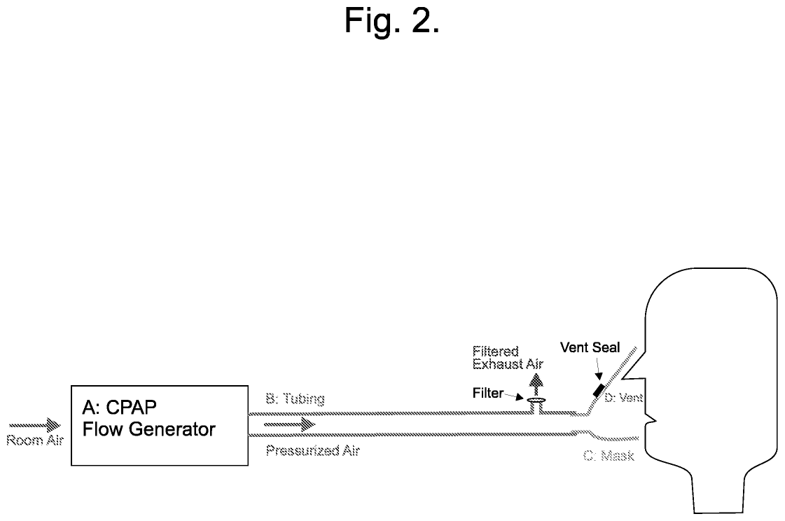

Referring first to FIG. 2, a basic embodiment of the system 100 is shown. A filter 160 may be placed over the Continuous Positive Airway Pressure (“CPAP”) mask 140 vent (e.g. by taping a filter fabric over the vent). Alternatively, a seal 149, or other impediment, may be placed over the non-filtered exhaust on the CPAP mask 140 by applying a commercial adhesive such as silicone or hot glue or caulk or other construction adhesive available at a hardware store (e.g. Home Depot). Alternatively, tape 149 or any other mechanism may also be used to seal the non-filtered exhaust on the CPAP mask.

An adapter 142 with a filtered exhaust 160 is added to the system (e.g. tubing or mask) near the patient. If the exhaust were added far from the patient it would increase the amount of “Dead Space” i.e. air the patient is ventilating (moving) that is not available for O2/CO2 exchange. The filtered exhaust prevents virus infected droplets from being dispersed into the room. The filtered exhaust may be created using the following or other methods: adding a T-shaped connector in-line with the tubing near the patient; or adding a new hole or using an existing hole (e.g. oxygen ports) on the patient mask to create a new exhaust channel; adding a filter to that new exhaust channel such as the Hudson RCI Bacterial Viral Filter model #1605. Alternatively, exhausted air may be channeled through a water seal system which includes a disinfectant.

The water seal system may be mechanically or ultrasonically agitated so that bubbles of air are broken down within the water seal chamber and exposed to the detergent in the water seal. The water seal system may then have a filter above the air above the fluid so that there is yet another level of removal of infection from droplets rising above the water system.

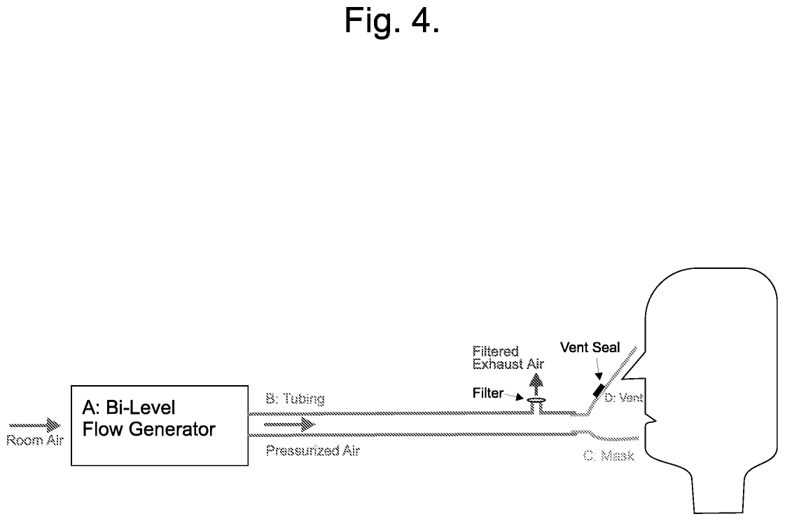

Turning now to FIG. 4, a filter 160 may be placed over the adapter outlet 146 positioned on the mask vent (e.g. by taping a filter fabric over the vent). Alternatively, a seal 149 may be placed over the non-filtered exhaust on the mask by applying a commercial adhesive such as silicone or hot glue or caulk or other construction adhesive available at a hardware store (e.g. Home Depot). Alternatively, tape 149 or any other mechanism may also be used to seal the non-filtered exhaust on the mask.

An exhaust 146 with a filter 160 is added to the system (e.g. tubing or mask) as an adapter 142 near the patient. If the exhaust were added far from the patient it would increase the amount of “Dead Space” i.e. air the patient is ventilating (moving) that is not available for 02/CO2 exchange. The filtered exhaust prevents virus infected droplets from being dispersed into the room. The filtered exhaust may be created using the following or other methods: adding a T-shaped connector in-line with the tubing near the patient; or adding a new hole or using an existing hole (e.g. oxygen ports) on the patient mask to create a new exhaust channel; adding a filter to that new exhaust channel such as the Hudson RCI Bacterial Viral Filter model #1605.

Alternatively, exhausted air may be channeled through a water seal system which includes a disinfectant. The water seal system may be mechanically or ultrasonically agitated so that bubbles of air are broken down within the water seal chamber and exposed to the detergent in the water seal. The water seal system may then have a filter above the air above the fluid so that there is yet another level of removal of infection from droplets rising above the water system.

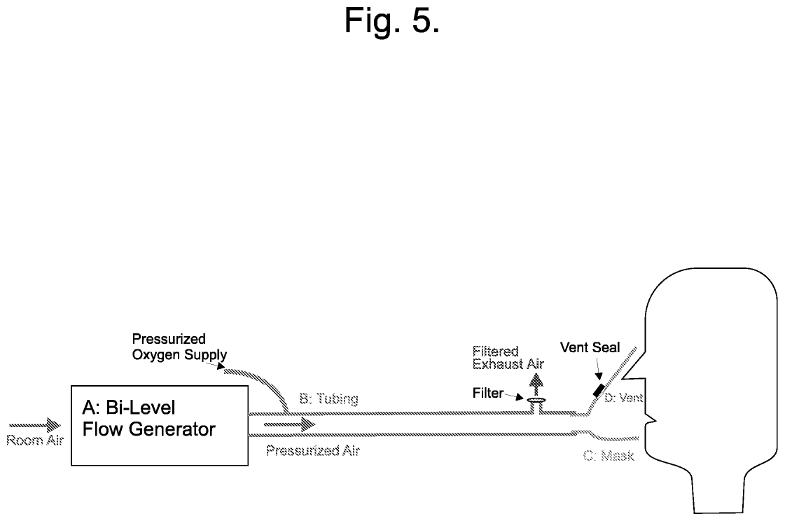

Turning now to FIG. 5, one may connect a pressurized oxygen supply, as available from a portable oxygen concentrator 150 or central supply in most hospitals, to the tubing 152 that leads to the channel 130.

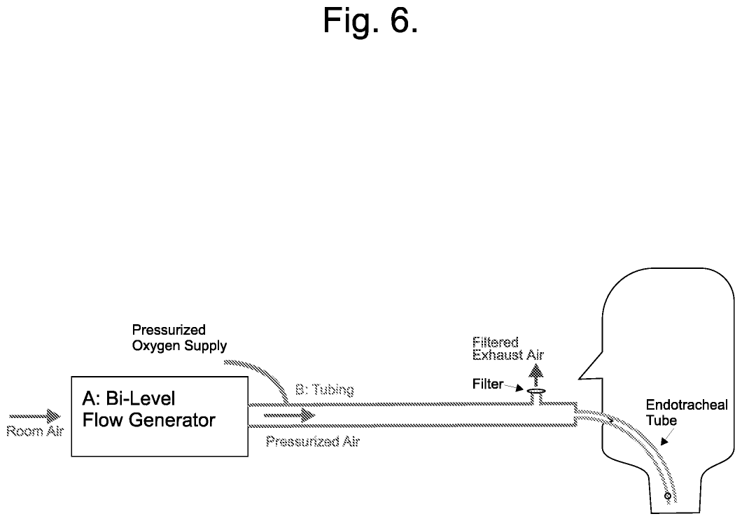

Turning now to FIG. 6, an airway protection device as a user interface 140 that precludes oral secretions or other materials from the oropharynx or nasopharynx from entering the trachea is placed between the channel 130 and the user's trachea. Connecting the channel from the bi-level PAP machine to an endotracheal tube or a laryngeal mask or a tracheostomy tube or other portal may aid in ventilating users' lungs more directly.

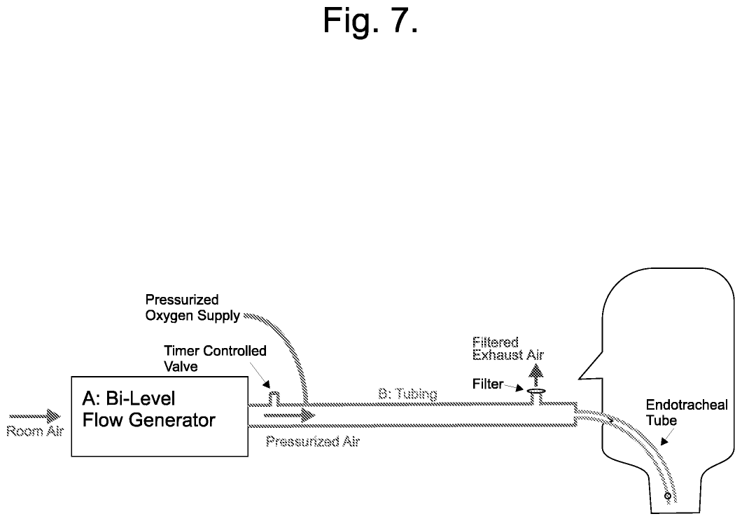

Turning now to FIG. 7, while some Bi-Level PAP machines may be programmed to have a “backup rate” whereby they initiate inspiration after a predefined number of seconds if the patient has not initiated inspiration spontaneously, the vast majority of bi-level PAP machines that have been sold over the last decade in the US or the world do not have this functionality. However, ordinary bi-level PAP machines can be triggered to initiate an inspiration by mimicking the subtle decrease in pressure that the machine normally senses when a patient spontaneously initiates a breath.

This subtle decrease in pressure can be mimicked by introducing a very brief (e.g. 100 msec) leak in the tubing circuit near the machine. If the leak is introduced near the motivator 120b, then the tube will not have any infected air that the patient had exhaled. To increase safety, a filter may 160 be placed around the area where the leak is created. One may place a T-shaped connector in-line with the tubing 130 near the machine 120b. One may place a valve 134 on the part of the “T” that is not in-line with the tubing (e.g. a solenoid valve or a rotating disc valve or other type of valve). One may place a controller 170 in communication with the valve 134 that opens the valve (e.g. for 100 ms to 2000 ms) at regular intervals (e.g. every 4 seconds) so that the bi-level PAP machine senses a decrease in pressure that triggers the bi-level PAP machine to initiate a breath. The controller may be built using a digital microprocessor controller or a simple timer chip such as the 555 timer chip introduced by SIGNETICS in 1972 or a mechanical timer.

Alternatively, a mechanical timer such as that used in a pulsating shower head may be used to intermittently allow and obstruct the leak (see e.g., U.S. Pat. No. 4,254,914, the contents of which are hereby incorporated by reference). Alternatively, a pressure transducer (e.g. piezoelectric crystal) operatively connected to a microcomputer 170 may be used to determine if the patient has spontaneously triggered a breath within the last four seconds (or some other time) and if not, then trigger a breath by opening the solenoid valve. The timer controlled valve may be powered by a battery or by splitting the power supply to the bi-level PAP machine. The microcontroller may be powered by a battery or by splitting the power supply to the bi-level PAP machine. So that a full breath is delivered when the valve opens, a Tₘᵢₙ minimum (minimum inspiratory time) and a Tmax maximum will need to be set on the bilevel flow generator (e.g. two seconds of minimum inspiratory time and two seconds of maximum inspiratory time).

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

### INDUSTRIAL APPLICABILITY

A novel method of reducing infection spread when a CPAP machine is used by a patient with an infection. This is especially important in nursing homes, hospitals, recovery rooms, emergency rooms and ICU settings to avoid infecting other healthcare workers or patients or surfaces.

A novel method of reducing infection spread when a BilevelPAP machine is used by a patient with an infection. This is especially important in nursing homes, hospitals, recovery rooms, emergency rooms and ICU settings to avoid infecting other healthcare workers or patients or surfaces.

A novel method of reducing infection spread when a BilevelPAP machine is used as a ventilator. This is especially important in nursing homes, hospitals, recovery rooms, emergency rooms and ICU settings to avoid infecting other healthcare workers or patients or surfaces.

A novel method of providing supplemental oxygen to patients using a bi-level PAP device converted to a ventilator.

A novel method of causing a BilevelPAP machine without a backup respiratory rate to have a backup respiratory rate.

What is claimed is:

1. A Continuous Positive Airway Pressure system for a user in an ambient environment, said machine comprising:
a gas motivator;
a gas channel, affixed to said gas motivator, to accept and conduct pressurized gas; and
a respiration interface, affixed to said gas channel both to accept pressurized gas and accept exhaled gas, dimensioned to sealingly connect to an airway of the user; and
an adapter comprising respiratory outlet having a filter obstructing an exclusive passage of said exhaled gas from the user to the ambient environment.

2. The system of claim 1 wherein said respiratory outlet is positioned on said interface.

3. The system of claim 1 wherein said respiratory outlet is positioned on said gas channel.

4. The system of claim 1 further comprising a liquid trap, between said respiratory outlet and the ambient environment, adapted to subject said exhaled gas to a barrier liquid.

5. The system of claim 5 wherein said liquid includes a disinfectant.

6. The system of claim 5 further comprising an agitator adapted to contact said barrier liquid for the agitation thereof.

7. The system of claim 1 wherein said interface includes original exhalation apertures sealingly covered.

8. The system of claim 1 wherein said respiratory outlet has a valve adapted to control a rate of exhausted air.

9. The system of claim 8 wherein said valve is adjustable to vary said rate of exhausted air.

10. An adapter for filtering exhalation of a user in an ambient environment, said adapter comprising:
an adapter body positioned between a gas motivator and a user interface, said body with a respiratory gas outlet having a filter obstructing an exclusive passage of exhaled gas from the user to the ambient environment.

11. The adapter of claim 10 further comprising an impediment array of at least one impediment adapted to seal pre-existing apertures between said ambient environment and the user to result in said exclusive passage.

12. A Continuous Positive Airway Pressure (“CPAP”) interface for a user in an ambient environment, said interface comprising:
an interface body, adapted to accept pressurized gas from a CPAP gas motivator, dimensioned to sealingly connect to an airway of the user; and
an adapter with a respiratory outlet having a filter obstructing an exclusive passage of exhaled gas from the user to the ambient environment.
